(19) United States
(12) Patent Application Publication
(10) Pub. No.: US 2023/0363767 A1
(43) Pub. Date: Nov. 16, 2023

(54) COMPRESSION FORCE INDICATOR FOR ACHIEVING HEMOSTASIS

(71) Applicant: Biolife, L.L.C., Sarasota, FL (US)

(72) Inventors: Talmadge Kelly Keene, Ruskin, FL (US); Kurt Vadelund, Apollo Beach, FL (US)

(21) Appl. No.: 18/078,551

(22) Filed: Dec. 9, 2022

Related U.S. Application Data
(60) Provisional application No. 63/287,867, filed on Dec. 9, 2021.

(51) Int. Cl. A61B 17/135

(52) U.S. Cl. CPC A61B 17/135; A61B 2017/12004

(57) ABSTRACT

This present device is a compression force indicating device for use during hemostasis. The device may be held in contact or close contact with the skin with the hand, by a circumferential band, a clamp or other mechanism and provides compression force data feedback to the clinician.

### RELATED APPLICATION

The present application claims the benefit of U.S. Provisional Patent Application No. 63/287,867, filed on Dec. 9, 2021, the entire disclosure of which is incorporated by reference herein.

### FIELD OF THE INVENTION

The present invention relates in general to devices and methods for determining the amount of compression over a wound to create hemostasis and, in particular, to devices and methods for achieving hemostasis of a blood vessel immediately after a vascular catheterization procedure.

### BACKGROUND OF THE INVENTION

Many medical procedures that once required extensive invasive surgery are performed today less evasively by inserting surgical or diagnostic devices through arteries or veins (i.e., vascular procedures, such as vascular catheterizations). These procedures are much safer and require significantly less recovery time than more evasive procedures performed years earlier before vascular techniques were developed. To prevent clots from forming in the vessels after surgery, the patient may require anticoagulation medications, which often results in excessive bleeding. To stop bleeding after vascular procedures, most clinics use pressure applied directly to the wound. This pressure must be held over both the entry point wound in the skin and the wound that was created in the vessel.

Direct pressure can be applied manually by a clinician, but in many instances the pressure is needed for an extended period of time to decrease or stop the bleeding. To save the clinicians time, many types of compression devices have entered the market. Examples of these devices are circumferential wraps designed to apply compression pressure over the wound and or artery, and clamp-like devices to apply the compression force.

Typical arterial pressure where many vascular procedures are performed is generally equal to the patient's blood pressure. This pressure is measured in millimeters of mercury (“mmHg”). 51.7 mmHg is equal to one pound per square inch (“psi”). A blood pressure of 80/120 mmHg is 1.5/2.3 psi. To close an artery by direct pressure compression for a patient with that blood pressure, a compression force or compression pressure of greater than 2.3 psi is all that is needed.

However, too much pressure will occlude the artery/vessel, and too little pressure will result in excessive bleeding or a hematoma. Ideal compression is termed “Patent Hemostasis.” Patent Hemostasis is sufficient pressure to stop bleeding at the skin and artery/vessel, but not so much pressure as to compress the artery/vessel closed.

Teaching a new clinician how much compression force to apply is often difficult. In addition, even if properly calculated and taught, the application of the necessary compression force by many circumferential devices is variable over time. In addition, the patient may often exhibit changes in blood pressure after vascular procedures. A simple device to provide appropriate compression force feedback so as to allow such compression devices to operate properly is needed in the medical industry.

The present invention addresses the long-felt need to accurately measure or observe the compression force applied to an artery so that Patent Hemostasis can be achieved.

### SUMMARY OF THE INVENTION

This invention relates generally to a device for measuring compression force to achieve hemostasis. The device employs a compressible portion with a rigid or semi-rigid bottom and an indicator of compression force that is visible to the clinician when applying a compression device.

Briefly, the present invention is directed, in an embodiment, to a device for improving hemostasis procedures, by providing the clinician direct feedback to the amount of compression force being applied to a wound site.

In certain embodiments, the indicator device may be a standalone device used in conjunction with adhesive strips, a hemostasis clamp, or a circumferential wrap.

In other embodiments, the indicator device may be mounted on or with a bracelet to operate as a circumferential compression device.

In other embodiments, to prevent the device from being dislodged from the hemostasis site, the indicator device may be held adjacent to the limb by a circumferential wrap and a strapping system used to apply compression force over the wound.

In other embodiments, the indicator device may be incorporated with an inflatable balloon device.

In other embodiments, the indicator device may employ a compressible polymer material, a balloon, a single spring, or two or more springs to create the compressible structure that applies the compression force.

### BRIEF DESCRIPTION OF THE DRAWINGS

FIG. 1 is a side cut-away view of a typical type of vascular access that needs compression to control hemostasis post-procedure.

FIG. 2 is a side view of an embodiment of the present invention having a compression device with indicator in an uncompressed condition.

FIG. 3 is side view of the compression device with indicator of FIG. 2.

FIG. 4 is top view of an embodiment of the compression indicator of the present invention showing the compression indicator gauge in both an uncompressed (short) condition and a compressed (lengthened) condition.

FIG. 5 top view of an alternative embodiment of the compression indicator of the present invention having indicator scale lines indicating a compressed (lengthened) condition.

FIG. 6 are top views of another embodiment of the compression indicator of the present invention having an indicator dial to indicate the amount of compression.

FIG. 7 is a top view of the inventive indicator of the present invention attached to a circumferential wrap or bracelet and having an indicator gauge with scale lines as shown in FIG. 5.

FIG. 8 is a side view of the inventive indicator of the present invention attached to a circumferential wrap or bracelet.

FIG. 9 is a side view of the inventive indicator of the present invention attached to a circumferential wrap or bracelet further having a tightening overwrap band that can be used to pull the inventive indicator over the wound.

FIGS. 10A and 10B are photographs of a prototype of the present inventive device.

FIG. 11 is a plan view of an alternative embodiment of the inventive indicator of the present invention shown in use on a patient.

### DETAILED DESCRIPTION OF PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

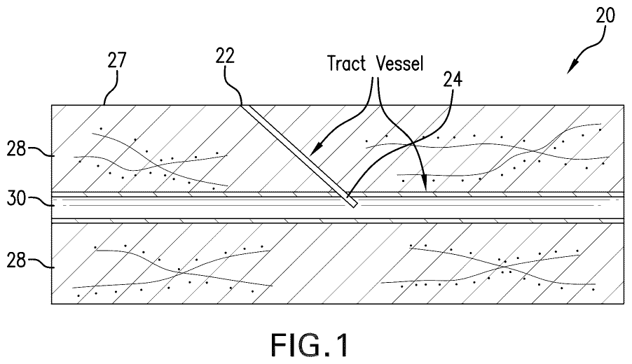

FIG. 1 illustrates a typical type of vascular access 20 through human tissue 28 that needs compression to control post-procedure hemostasis. Compression must control bleeding at the wound in blood vessel 30 and at the wound 22 in the skin 27. A typical insertion track 24 through human tissue 28 is made into blood vessel 30 by insertion to access a vein or artery 30.

As shown in FIG. 1, the insertion wound 22 at skin 27 is often not aligned vertically with the vascular wound entering blood vessel 30. This difference in vertical alignment typically increases with depth of the vessel. Therefore, different sized and shaped structures may be necessary to meet the variety of medical application needs.

Compression must be held in place to simultaneously control bleeding at the dermatotomy (skin wound) 22 and the arteriotomy (wound in the artery or vessel) 24. The ideal pressure is Patent Hemostasis pressure, which is sufficient pressure to obtain hemostasis at the arteriotomy, but still allow some blood flow through the artery or vein 30. Patent Hemostasis pressure is thought to be approximately mean arterial pressure or “MAP”.

The indicator device of the present invention is designed such that a vertical change in the device provides a horizontal indication of compression force.

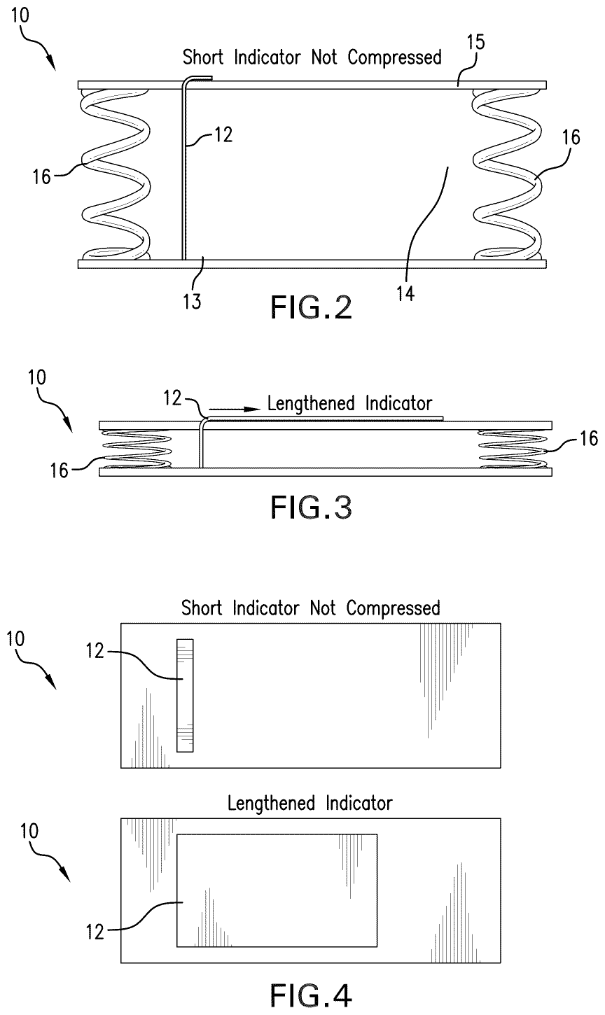

FIG. 2 shows an inventive indicator device 10 when not compressed. As device 10 is compressed, flexible indicator 12 will push upwards and then be forced horizontally. The amount of downward force will determine how far indicator 12 moves horizontally. The bottom portion (the portion that contacts the patient) 13 would be deemed the foot, the center portion 14 is the compressible middle (springs 16), and the top 15 features the viewing part of the device and may have a clear cover and a flexible material designed to move horizontally as the device is compressed vertically.

The top and bottom of indicator device 10 may be made of any material, including polymeric material, as known in the art, provided it has sufficient rigidity to maintain compression when compression force is applied to the wound sites.

FIG. 3 illustrates the inventive indicator device 10 of FIG. 3 in a compressed state wherein the flexible indicator 12 is lengthened as the springs 16 are compressed.

FIG. 4 is a top view of the inventive device 10. Again, the amount that flexible indicator 12 moves horizontally will be proportional to the amount of compression.

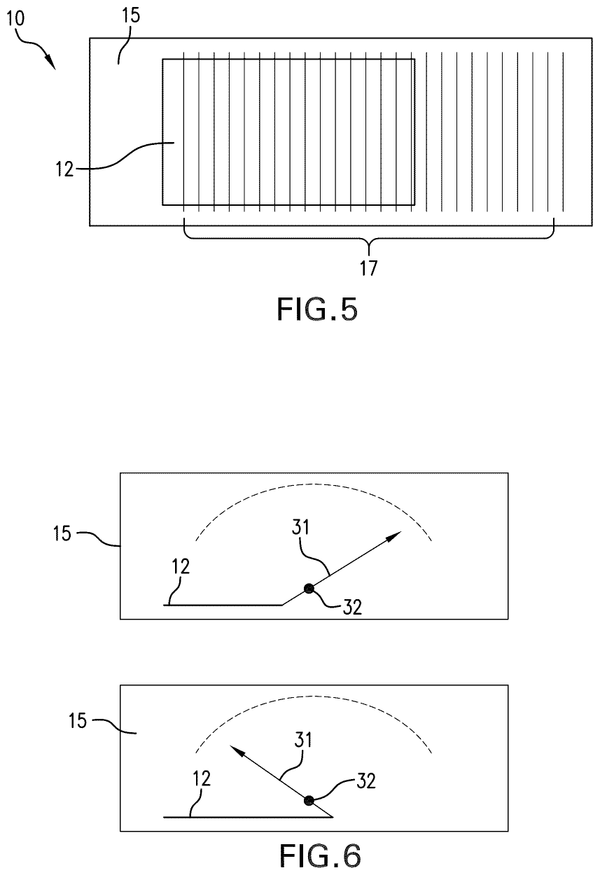

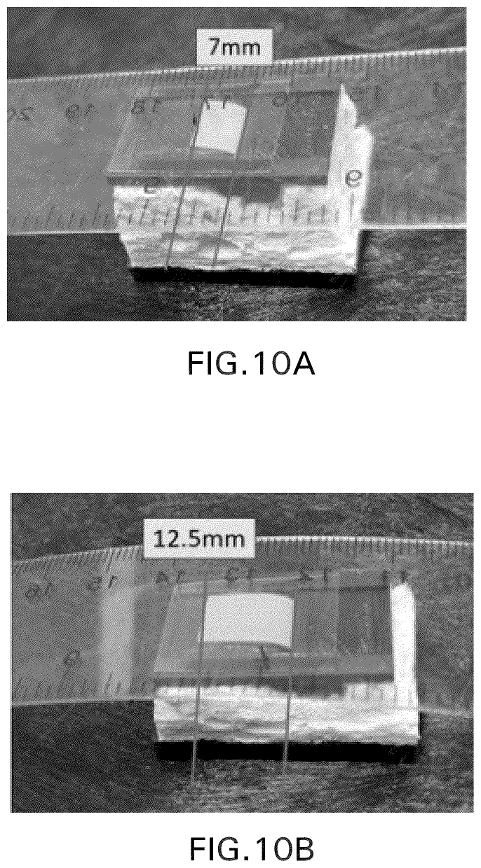

A marked scale 17, as shown in FIG. 5, may be applied to the top 15 of the indicator device 10 to measure the amount of compression. In other embodiments (not shown), flexible indicator 12 may be marked with a scale or may have different colors to indicate amount of movement. As such, various embodiments to provide the necessary visual feedback may include a number scale related to mmHg, or a color scale, or both, or any another feedback type device, including a digital feedback system (shown in FIG. 10) to indicate to the clinician how much compression force is being applied to the bleeding site.

It may be that that the compression plates (bottom 13 and top 15) do not create sufficient vertical movement to use a direct measurement system. In that case, a movement magnifying system, such as shown in FIG. 6, may be necessary. In this FIG. 6, a lever 31 and a fulcrum or pivot point 32 are used to magnify the horizonal movement created by the vertical compression of the device. Again, the vertical movement of the overall device translates to a horizontal movement of the flexible material that in turn moves the indicator needle or dial in this sketch.

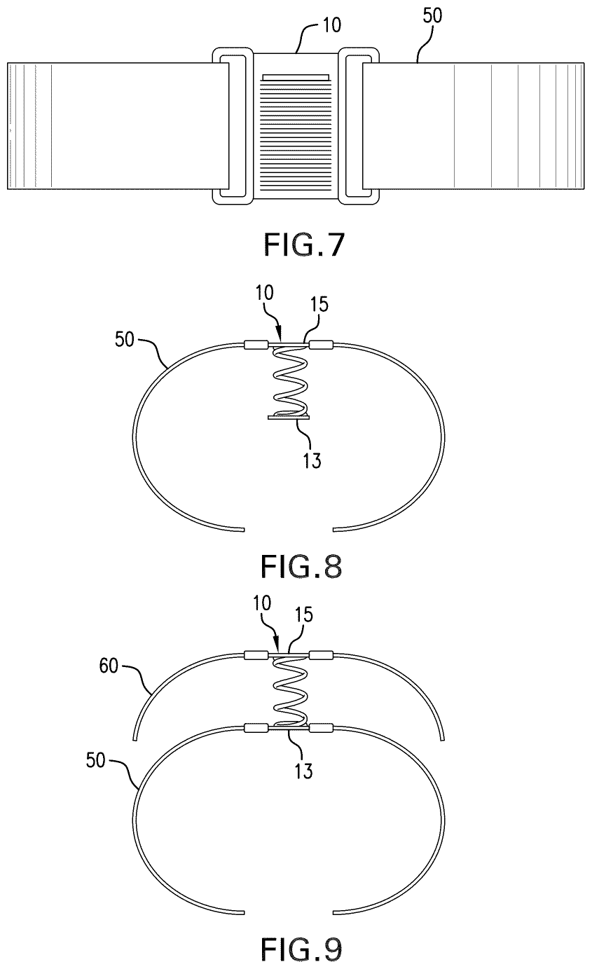

FIGS. 7 and 8 illustrate the inventive indicator device 10 attached to a circumferential wrap or bracelet 50. The wrap 50 may use any number of secure options. For example, such securement means may include, but are not limited to, Velcro or other hook and loop attachment, an adhesive to the skin or onto itself, buttons, magnets, or a zip-tie mechanism. The wrap 50 may connect to the other side of the wrap or be a single wrap that is pulled through the device and secured to itself.

FIG. 9 illustrates an overwrap band 60 that holds the device over the wound, and that can then be tightened by an overwrap used to pull the device bottom 13 down, applying indirect compression force over the wound. The device is designed to allow it to slide down from the housing held by the circumferential wrap.

The device may be of any shape designed to fit the site on the patient where hemostasis is needed.

The device may use a metal spring or multiple springs as the compressible material between the rigid/semi-rigid top 15 and bottom 13. It may also employ an air bladder, a foam, or silicone as the compressible material. Any compressible material may be used provided it is consistent and can be calibrated to provide usable feedback to the clinician. The bladder may be semi-rigid and prefilled with a compressible liquid (air or gas) or the bladder may be designed to expand. The bladder may also be designed to be inflated with a syringe.

Typically, a direct measurement of mass (lbs, kg, etc) is not sufficient and a clinician will want mmHg to coincide with blood pressure. Therefore, the surface area of the foot or skin contact portion of the device must be considered for the tick marks or measurements. It would be possible to create multiple housings that use the same compression indicator device. The compression force markings would be on the housing, with the indicator on the insert.

The main advantage of this design is that the vertical movement is translated to a horizontal movement of the indicator by use of a compressible, flexible material. A pure vertical movement indicator would create a profile that is too high and result in dislodgement by patients. The compression needs to be maintained directly over the dermatotomy and arteriotomy. This unique product creates a sleek design and functional product.

### EXAMPLES

### Example 1

A 3D printer was employed to create a prototype of the present inventive device. FIGS. 10A and 10B are photographs showing a yellow sponge, flexible tape measure, and clear slide cover to create the viewing window were employed. The tape was glued to the bottom or foot, a hole for the tape was cut in the sponge, and the top portion included a rounded slit to turn the tape from vertical to horizontal. As the device was compressed vertically, the indicating tape lengthened.

For this example, the compression indicator was used beneath a clear wrap (3 wraps were needed) to simulate hemostasis post radial artery access. As the device was compressed the indicator tape lengthened. In this example, 1.95 lbs of pressure was applied over 1 square inch, so approximately 100 mmHg was applied to the simulated wound site. This amount of compression would create patent hemostasis for most patients. Without the inventive indicator, it would be impossible to know how much compression is being applied.

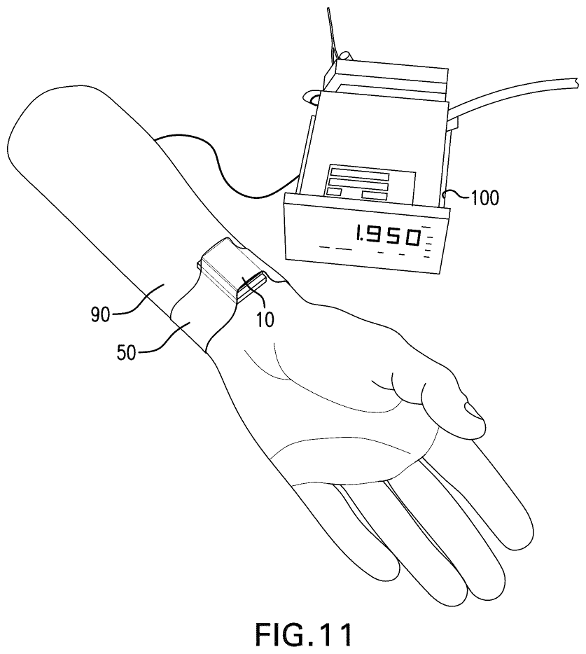

FIG. 11 shows the inventive indicator device 10 mounted on a bracelet 50 attached to a human wrist 90 and using a digital read-out monitor 100.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the versions contained therein.

What is claimed is:

1. A compression indicating device for hemostasis comprising a rigid or semi-rigid foot, a middle compressible portion, a top viewing portion and a flexible material that turns horizontally with vertical movement as the device is being compressed to provide compression force feedback to user of the indicating device.

2. The compression indicating device of claim 1 wherein the middle compressible portion comprises a single spring.

3. The compression indicating device of claim 1 wherein the middle compressible portion comprises multiple springs.

4. The compression indicating device of claim 1 wherein the middle compressible portion comprises a foam, silicone or some other compressible material.

5. The compression indicating device of claim 1 wherein the middle compressible portion comprises an air bladder.

6. The compression indicating device of claim 1 wherein the top viewing portion comprises indicating measurements or indicating colors.

7. The compression indicating device of claim 1 wherein the flexible material comprises indicating measurements or indicating colors.

8. The compression indicating device of claim 1 further comprising a lever with a fulcrum attached to the flexible material to magnify the vertical compression of the compression indicating device.

9. A circumferential wrap for applying hemostasis compression comprising the compression indicating device of claim 1.

10. The compression indicating device of claim 1 further comprising medication selected from the group consisting of antimicrobials, hemostatic materials, or other wound treatment materials.

11. The compression indicating device of claim 1 wherein said compression indicating device is sized to fit inside of multiple housings to coincide with different surface area sized or shaped housing feet and to ensure close contact of the middle compressible portion with such housing feet.

12. The compression indicating device of claim 1 wherein the middle compressible portion comprises an inflatable bladder.

13. A method of using the compression indicating device of claim 1 to create patent hemostasis pressure over a wound site to control bleeding of an arterial access comprising the steps of:
a. placing the compression indicating device over the wound site;
b. applying compression to the device to create a pressure at the wound site;
c. reading the data generated by the compression indicating device to determine whether the compression applied to the device should be altered; and
d. alternatively, altering the amount of compression applied to the device to achieve hemostasis at the wound site.
